(19) United States
(12) Patent Application Publication
(10) Pub. No.: US 2006/0269616 A1
(43) Pub. Date: Nov. 30, 2006

(54) Supplement composition and method of use for enhancement of DNA repair process

(75) Inventor: Vincent C. Giampapa, Little Falls, NJ (US)

Correspondence Address: MELVIN K. SILVERMAN, 500 WEST CYPRESS CREEK ROAD, SUITE 500, FT. LAUDERDALE, FL, 33309, US

(73) Assignee: Suracell, Inc., Montclair, NJ (US)

(21) Appl. No.: 11/439,641

(22) Filed: May 24, 2006

Related U.S. Application Data
(60) Provisional application No. 60/685,143, filed on May 26, 2005.

(51) Int. Cl. A61K 36/185; A61K 31/70; A61K 31/015; A61K 31/455

(52) U.S. Cl. 424/641; 424/725; 514/23; 514/356; 514/763

(57) ABSTRACT

A supplement composition for enhancing DNA repair process is provided, which contains a carotenoid compound, a nicotinamide compound, a zinc compound, D-ribose, and a water soluble extract of an Uncaria species. The composition can further contain resveratrol and a glucosinolate compound. Further provided is a method of using the supplement composition for enhancing an individual's DNA repair process.

### CROSS REFERENCE TO RELATED APPLICATION

This application claims the benefit Under 35 USC 119 (e) of the provisional patent application Ser. No. 60/685,143, filed May 26, 2005, which is herein incorporated by reference in its entirety.

### FIELD OF THE INVENTION

The present invention relates to dietary supplement compositions and the method of use thereof for enhancement of the DNA repair process.

### BACKGROUND OF THE INVENTION

DNA repair is a process constantly operating in cells. It is essential to survival because it protects the genome from damage and harmful mutations. In human cells, both normal metabolic activities and environmental factors (such as UV rays) can cause DNA damage, resulting in as many as 500,000 individual molecular lesions per cell per day. These lesions cause structural damage to the DNA molecule, and can dramatically alter the cell's way of reading the information encoded in its genes. Consequently, the DNA repair process must be constantly operating, to correct rapidly any damage in the DNA structure.

As cells age, however, the rate of DNA repair decreases until it can no longer keep up with ongoing DNA damage. The cell then suffers one of three possible fates: an irreversible state of dormancy, known as senescence; cell suicide, also known as apoptosis or programmed cell death; carcinogenesis, or the formation of cancer. Most cells in the body first become senescent. Then, after irreparable DNA damage, apoptosis occurs. In this case, apoptosis functions as a “last resort” mechanism to prevent a cell from becoming carcinogenic and endangering the organism. When cells become senescent, alterations in biosynthesis and turnover cause them to function less efficiently, which inevitably causes disease. The DNA repair ability of a cell is vital to the integrity of its genome and thus to its normal functioning and that of the organism. Many genes that were initially shown to influence lifespan have turned out to be involved in DNA damage repair and protection.

Various nutritional substances have been found useful in support of DNA repair process. Vitamin A is a family of fat-soluble vitamins. Retinol is one of the most active, or usable, forms of vitamin A. Carotenoids such as beta carotene, sometimes called pro-vitamin A, are water-soluble precursors which are converted to vitamin A by the body. Lycopene, lutein, and zeaxanthin are other carotenoids commonly found in food. The exact mechanism of action of carotenoids such as beta carotene is not fully understood but it is commonly accepted scientifically that one primary mechanism is to scavenge oxygen derived free radicals produced either as by-products of metabolism or from exogenous environmental exposures. As free radicals scavenger, carotenoids can be expected to reduce or protect against the chemical damage induced in DNA, RNA and protein of cells by toxic environmental exposures or endogenous cellular metabolic errors that ultimately can result in a disease state.

Nicotinamide and its metabolic equivalent nicotinic acid (niacin, vitamin B₃) or even tryptophane which is the synthetic precursor to niacin, is the main precursor for the formation and maintenance of the cellular pool of nicotinamide adenine dinucleotide (NAD). NAD is essential for cellular ATP production and maintenance of the cell's redox potential, and it is also the substrate for the DNA repair enzyme, poly ADP-ribosyl transferase (ADPRT). Niacin deprivation decreases the NAD pool significantly both in tissue culture cells, animal systems and humans. The NAD depleted cells have an increased sensitivity to DNA damage, and the levels of poly(ADP-ribose) production in cultured cells or in rat liver were significantly lower after mild nicotinamide deficiency. On the other hand, when niacin was given as a supplement to ordinary nutrition (i.e. above known dietary levels) the NAD pool increased and the cells were less sensitive to oxygen radicals. Therefore, the primary mechanism of action of nicotinamide/niacin differs from carotenoids in that the cell's potential for energy metabolism is increased by amplifying NAD and ATP pool supplies, which in turn is useful to cells, tissues and organs to reduce DNA damage, enhance DNA repair (i.e. poly ADP-ribosylation) and stimulate immune function.

Zinc differs from the carotenoids and nicotinamide with regard to its mechanism of action in that it influences disease development and immune function by being an essential co-factor in several enzyme functions involving replication, DNA repair and antioxidant defense of cells. Zinc is required for cell replication and DNA polymerase activity. There are two zinc fingers in the DNA binding domain of the ADPRT gene and other DNA repair proteins which contain cysteine residues, and if these cysteine residues are oxidized at their thiol constituents, they would prevent DNA binding and participation in DNA repair. Moreover, superoxide dismutase is an antioxidant enzyme protecting cells from the harmful superoxide anion because this radical is a substrate for the enzymatic reaction that also requires zinc as a cofactor.

U.S. Pat. No. 6,020,351 (to Pero) teaches the use of a combination of carotenoids, nicotinamide, and zinc, in the absence of other active components, to reduce DNA damage, enhance DNA repair capacity, and enhance immune function. Commercially, a combination of these three materials is available under the tradename of Nicoplex®.

As taught in U.S. Pat. Nos. 6,039,949, 6,238,675, 6,361,805 and 6,964,784, the water soluble extract of an Uncaria species, and its bioactive components, carboxy alkyl esters, is known to give profound nutritional support as a dietary supplement because the water soluble extract of an Uncaria species enhances DNA repair process and immune functions. Both of these processes involve regulating the nuclear transcription kappa beta (NF-kB). NF-kB is well known to control the nuclear events that salvage cells from apoptotic cell death and pro-inflammatory cytokine production. Hence, this mechanism directly connects induction of apoptosis to programmed cell toxicity with inhibition of pro-inflammatory cytokine production and inflammation.

Resveratrol has been reported having potent antioxidant activity and having the ability to inhibit platelet aggregation. It is also thought to play a significant role in the repair and maintenance of DNA strands. Research studies have also found that resveratrol is able to block all three mechanisms of cancer formation by helping the body inhibit tumor initiation, promotion and progression. Resveratrol has been used recently for caloric mimic therapy.

Indole-3-carbinol is a naturally occurring phytochemical found in cruciferous vegetables such as cabbage, broccoli, and kale. It is a member of the class of sulfur-containing chemicals called glucosinolates. Indole-3-carbinol and other glucosinolates, for example, other indoles and isothio-cyanates such as sulforaphane, are antioxidants and potent stimulators of natural detoxifying enzymes in the body. Indole-3-carbinol and other glucosinolates are believed to be responsible for the lowered risk of cancer in humans that is associated with the consumption of broccoli and other cruciferous vegetables. Indole-3-carbinol is also known to support the liver's detoxification processes as well as normal cellular reproduction.

D-ribose is a five-carbon sugar (pentose) found primarily in ribonucleic acid, and it is a naturally occurring sugar found in all living cells. Some of the most important biological molecules contain D-ribose, including ATP (adenosine triphosphate), all the nucleotides and nucleotide coenzymes and all forms of RNA (ribonucleic acid).

D-Ribose is used to generate ATP (adenosine triphosphate). In theory, supplementing diet with additional ribose should increase the rate at which ATP is generated, leading to an improvement in exercise performance and faster muscle growth. D-ribose, when taken as a nutritional supplement, bypasses the slow conversion steps needed to recreate the adenosine nucleotide, and is readily available for the creation of more ATP. Therefore, supplemental ribose can increase the speed at which these nucleotides are replaced. D-ribose increases both de novo (new) synthesis and salvage of nucleotides in heart and skeletal muscle. Heart and skeletal muscle cells cannot quickly replace lost nucleotides due to low availability of two rate limiting enzymes in the Pentose Phosphate Pathway. Supplemental ribose bypasses the rate limiting steps of the Pentose Phosphate Pathway, speeding syntheses of nucleotides that may be lost from the cell through catabolism and salvage of nucleotides. Some recent research suggests that supraphysiological amounts of this sugar may have cardioprotective effects, particularly for the ischemic heart.

The above-described nutrients have been used individually, or in a partial combination, for enhancing DNA repair, or other functions. It is desirable to provide a dietary supplement composition including these active components, which function synergetically to provide further enhancement of DNA repair process.

### SUMMARY OF THE INVENTION

In one embodiment, the present invention is directed to a supplement composition for enhancing an individual's DNA repair process. The supplement composition comprises a carotenoid compound, a nicotinamide compound, a zinc compound, D-ribose, and a water soluble extract of an Uncaria species. In a further embodiment, the supplement composition further comprises effective amounts of a glucosinolate compound and resveratrol. In an alternative embodiment, the supplement composition comprises a carotenoid compound, a nicotinamide compound, a zinc compound, a glucosinolate compound and resveratrol and a water soluble extract of an Uncaria species. Moreover, the supplement composition also comprises pharmaceutically acceptable excipients, and can be provided in a form for oral administration.

In a further aspect, the present invention is directed to a method for enhancement of an individual's DNA repair process. In one embodiment, as a regular strength treatment, the method comprises administering a supplement composition to a person daily in a dosage comprising from about 2,500 IU to about 10,000 IU of beta-carotene, from about 75 mg to about 500 mg of niacinamide, from about 5 mg to about 20 mg of zinc citrate, from about 75 mg to about 500 mg of D-ribose, and from about 85 mg to about 350 mg of water soluble extract of an Uncaria species.

In a further embodiment, as an extra strength treatment, the method comprises administering a supplement composition to a person daily in a dosage comprising from about 2,500 IU to about 10,000 IU of beta-carotene, from about 75 mg to about 500 mg of niacinamide, from about 5 mg to about 20 mg of zinc citrate, from about 100 mg to about 400 mg of indole-3-carbinol, from about 10 mg to about 40 mg of resveratrol, and from about 85 mg to about 350 mg of water soluble extract of an Uncaria species. Moreover, in the extra strength treatment, the supplement composition can further comprise from about 75 mg to about 500 mg of D-ribose in the daily dosage.

### DETAILED DESCRIPTION OF THE INVENTION

In one embodiment, the present invention provides a supplement composition which comprises a carotenoid compound, a nicotinamide compound, a zinc compound, D-ribose, a water soluble extract of an Uncaria species, and pharmaceutically acceptable excipients for oral administration to enhance DNA repair process, and increase an individual's resistance to DNA damages.

Carotenoids are electrophilic scavenger of radicals produced endogenously by cells or exogenously by the environment. Nicotinamide is amplified source of energy via increased production of NAD or ATP. Zinc is an essential cofactor in several enzyme functions involving replication, DNA repair and antioxidant defense of cells. The water soluble extract of an Uncaria species prevents free radical damage by NF-kB inhibition, induces differentiation and immune cell responsiveness by apoptosis, enhances DNA repair, and inhibit tumor cell growth, which in turn are the major factors related to aging. Resveratrol has potent antioxidant activity and plays a significant role in the repair and maintenance of DNA strands. Indole-3-carbinol is antioxidant and potent stimulator of natural detoxifying enzymes in the body. In combination, these six nutrients can synergistically achieve improvement of resistance to DNA damage, enhancement of DNA repair capacity and immune cell responsiveness, and hence reduce an individual's likelihood of developing aging related disorders.

The zinc compound, as used herein, includes an appropriate source of zinc for administration to humans and/or animals, for example, one or more zinc salts, such as zinc citrate, halides, nitrates, sulfates, oxides or acetates, or amino acids such as methionine or aspartate, dipeptides, or gluconates. In one embodiment of the present invention, the composition comprises zinc citrate, preferably in an amount ranging from about 5 mg to about 20 mg in one dosage. In one preferred embodiment, the composition comprises about 10 mg of zinc citrate in one dosage.

Herein, one dosage is also referred to as one serving. If the supplement composition is provided in the form of tablet, one dosage can be either one tablet, or two to three tablets. For example, 10 mg of zinc citrate in one dosage can be provided in one tablet as shown in Supplement Composition A of Example 1, and 10 mg of zinc citrate in one dosage can be provided in two tablets as shown in Supplement Composition B of Example 2, wherein each tablet contains 5 mg of zinc citrate. The size and number of tablets may depend on the manufacturability, which may further depend on the properties of the components and the pharmaceutically acceptable excipients used.

The carotenoid compound, as used herein, includes carotenoids, such as alpha carotene, beta carotene, gamma carotene, lycopene or combination thereof. Preferably, beta-carotene is used in the supplement composition, because beta-carotene is a carotenoid that is more efficiently converted to retinol than other carotenoids. In one embodiment of the present invention, the composition comprises beta-carotene, preferably in an amount ranging from about 2,500 to about 10,000 international unit (IU) in one dosage. In one preferred embodiment, the supplement composition comprises about 5,000 IU of beta-carotene in one dosage.

The nicotinamide compound, as used herein, includes nicotinamide, niacin, tryptophane, NAD (nicotinamide-adenine dinucleotide), NADH (reduced form of NAD), NADP (NAD phosphate), NADPH (reduced form of NADP) or combination thereof. In one embodiment of the present invention, the supplement composition comprises nicotinamide, preferably in an amount ranging from about 125 mg to about 500 mg in one dosage. In one preferred embodiment, the supplement composition comprises about 250 mg of nicotinamide in one dosage.

D-ribose is a sweet, solid, water-soluble substance that is also known as alpha-D-ribofuranoside. Supplemental D-ribose is produced from the fermentation of corn syrup. In one embodiment of the present invention, the composition comprises D-ribose, preferably in an amount ranging from about 75 mg to about 500 mg in one dosage. In one preferred embodiment, the composition comprises about 150 mg of D-ribose in one dosage. In another preferred embodiment, the composition comprises about 250 mg of D-ribose in one dosage.

The Uncaria species includes tomentosa, guianensis, pteropoda, homomalla, perrottetii, or rhynchopylla. The term “water soluble extract of an Uncaria species” used herein refers to the water soluble extract of an Uncaria species obtained using the method described in U.S. Pat. Nos. 6,361,805, 6,238,675 and 6,039,949, which are hereby incorporated by reference in their entirety. Furthermore, the bioactive active component of the water soluble extract of an Uncaria species has been identified as carboxy alkyl esters, as described in U.S. Pat. No. 6,964,784, which is hereby incorporated by reference in its entirety.

The water soluble extract of an Uncaria species is commercially available under the trade name Activar AC-11®, or C-Med-100®, from Optigenex, Inc, New York, N.Y. More specifically, Activar AC-11® is a hot water extract from the bark of Uncaria tomentosa, produced according to the process described in U.S. Pat. No. 6,039,949. Briefly, the extract is produced from heating 150 gm of bark in 5 liters of tap water for 12 hours at 95° C., decanting the soluble fraction, ultra-filtrating the resulting water extract to remove all components having molecular weight larger than 10,000 daltons. The fraction having molecular weight less than 10,000 daltons is spray dried. The product is in a form of beige to brown-orange hygroscopic fine powder, and it contains no less than 16% of carboxy alkyl esters, less than 0.05% of indole alkaloids of less than 10,000 daltons and 0% of indole alkaloids of higher than 10,000 daltons, and it is readily soluble in water (solubility in water >400 mg/ml). In one embodiment of the present invention, the composition comprises the water soluble extract of the Uncaria species in the form of Activar AC-11®, preferably in an amount ranging from about 85 mg to about 350 mg in one dosage. In one preferred embodiment, the composition comprises about 175 mg of Activar AC-11® in one dosage.

In a further embodiment, the supplement composition of the present invention further comprises a glucosinolate compound, and resveratrol.

The glucosinolate compound, as used herein, includes indole-3-carbinol, and other indoles, and isothiocyanates such as sulforaphane. In one embodiment of the present invention, the supplement composition comprises indole-3-carbinol, preferably in an amount ranging from about 100 mg to about 400 mg in one dosage. In a preferred embodiment, the supplement composition comprises about 200 mg of indole-3-carbinol in one dosage.

Resveratrol is a unique compound produced by the skins of grapes, grapevines and other plants and their roots in response to environmental stresses. In one embodiment of the present invention, the composition comprises resveratrol, preferably in an amount ranging from about 10 mg to about 40 mg in one dosage. In one preferred embodiment, the composition comprises about 20 mg of resveratrol in one dosage.

All above described active components used in the illustrative, or preferred embodiments such as beta carotene, nicotinamide, zinc citrate, resveratrol, indole-3-carbinol, D-ribose, and Activar AC-11® are commercially available.

Example 1 illustrates an exemplary supplement composition of the present invention, which is considered as a regular strength formula. Examples 2 and 3 illustrate two exemplary supplement compositions of the present invention, which are considered as extra strength formulas. As shown in Example 2, in the extra strength formula, the supplement composition comprises beta carotene, nicotinamide, zinc citrate, indole-3-carbinol, resveratrol, and water soluble extract of an Uncaria species. As further shown in Example 3, the extra strength formula can further comprise D-ribose.

As a convenient form of dietary supplement, the supplement compositions described above are provided in the form of tablet. However, it should be understood that tablet is only one of various convenient dosage forms which can be used for the supplement composition. Other suitable forms include hard or soft-gelatin capsules, powders, or in liquid dosage forms, such as elixirs, syrups, dispersed powders or granules, emulsions, or aqueous or oily suspensions. When other dosage forms are used, the amounts of the active components in one dosage remain the same, however, the concentration of the component in different pharmaceutical media can be different.

Preferably, the supplement composition is formulated as a tablet, and as such it can contain pharmaceutically acceptable excipients, according to methods and procedures well known in the art. As used herein, “excipients” means substances that are of little or no therapeutic value, but useful in the manufacture and compounding of various pharmaceutical preparations, which form the medium of the supplement composition. These substances include coloring, flavoring, and diluting agents; emulsifying and suspending agents; ointment bases; pharmaceutical solvents; antioxidants and preservatives for the product; and miscellaneous agents. Suitable excipients are described in Remington's Pharmaceutical Sciences, Mack Publishing Company, a standard reference text in this field, which is incorporated herein by reference in its entirety.

As used herein, “tablets” are solid pharmaceutical dosage forms containing active ingredients with or without suitable diluents and prepared either by compression or molding methods well known in the art. Although tablets are most frequently discoid in shape, they may also be round, oval, oblong, cylindrical, or triangular. They may differ greatly in size and weight depending on the amount of active ingredients present and the intended method of administration. They are divided into two general classes, (1) compressed tablets, and (2) molded tablets or tablet triturates. In addition to the active ingredients, tablets contain a number of inert excipients or additives. A first group of such excipients includes those materials that help to impart satisfactory compression characteristics to the formulation, including diluents, binders, and lubricants. A second group of such excipients helps to give additional desirable physical characteristics to the finished tablet, such as disintegrators, colors, flavors, and sweetening agents. Compressed tablets can be uncoated or can be sugar coated or film coated by known techniques to mask any unpleasant taste and protect the tablet from the atmosphere, or enteric coated for selective disintegration and adsorption in the gastrointestinal tract.

As used herein, “diluents” are inert substances added to increase the bulk of the formulation to make the tablet a practical size for compression. Commonly used diluents include calcium phosphate, calcium sulfate, lactose, kaolin, mannitol, sodium chloride, dry starch, powdered sugar, silica, and other suitable materials. As used herein, “binders” are agents used to impart cohesive qualities to the powdered material. Binders insure the tablet remaining intact after compression, as well as improving the free-flowing qualities by the formulation of granules of desired hardness and size. Materials commonly used as binders include starch; gelatin; sugars, such as sucrose, glucose, dextrose, molasses, and lactose; natural and synthetic gums, such as acacia, sodium alginate, extract of Irish moss, panwar gum, ghatti gum, mucilage of isapol husks, carboxymethylcellulose, methylcellulose, polyvinylpyrrolidone, Veegum, microcrystalline cellulose, microcrystalline dextrose, amylose, and larch arabogalactan, and other suitable materials. As used herein, “lubricants” are materials that perform a number of functions in tablet manufacture, such as improving the rate of flow of the tablet granulation, preventing adhesion of the tablet material to the surface of the dies and punches, reducing interparticle friction, and facilitating the ejection of the tablets from the die cavity. Commonly used lubricants include talc, magnesium stearate, calcium stearate, stearic acid, and hydrogenated vegetable oils. As used herein, “coloring agents” are chemicals that give tablets a more pleasing appearance, and in addition help the manufacturer to control the product during its preparation and help the user to identify the product. Any of the approved certified water-soluble FD&C dyes, mixtures thereof, can be used to color tablets.

In a further aspect, the present invention provides the method of using the supplement compositions described above as a dietary supplement to individuals, particularly those in need thereof. Preferably, the supplement composition is administrated daily. In one embodiment, as a regular strength treatment, the preferred daily dosage includes from about 2,500 IU to about 10,000 IU of beta-carotene, from about 75 mg to about 500 mg of niacinamide, from about 5 mg to about 20 mg of zinc citrate, and from about 75 mg to about 500 mg of D-ribose, and from about 85 mg to about 350 mg of water soluble extract of an Uncaria species.

In another embodiment, as an extra strength treatment, the preferred daily dosage includes from about 2,500 IU to about 10,000 IU of beta-carotene, from about 75 mg to about 500 mg of niacinamide, from about 5 mg to about 20 mg of zinc citrate, from about 85 mg to about 350 mg of water soluble extract of an Uncaria species, from about 100 mg to about 400 mg of indole-3-carbinol and from about 10 mg to about 40 mg of resveratrol. Furthermore, the daily dosage can further include from about 75 mg to about 500 mg of D-ribose.

Supplement Composition A of Example 1 is considered as a regular strength formula for enhancing DNA repair process. The preferred daily dosage is one tablet of Supplement Composition A, which contains about 5,000 IU of beta-carotene, about 150 mg of niacinamide, about 10 mg of zinc citrate, about 150 mg of D-ribose, and about 175 mg of the water soluble extract of an Uncaria species. Supplement Composition B of Example 2 and Supplement Composition C of Example 3 are considered as extra strength formulas, which are particularly suitable for individuals who need enhancement of DNA repair because of genetic predisposition, clinical conditions, advanced aging, or poor environmental impact. For these individuals, a preferred daily dosage is two tablets of Supplement Composition B which contains about 5,000 IU of beta-carotene, about 250 mg of niacinamide, about 10 mg of zinc citrate, about 200 mg of indole-3-carbinol, about 20 mg of resveratrol, and about 175 mg of the water soluble extract of an Uncaria species; or two tablets of Supplement Composition C of Example 3, which further contains about 250 mg of D-ribose.

A method of determining an individual's genetic predisposition in DNA repair is described in co-pending patent application Ser. No. 60/796,423 entitled “Method of Determining Genetic Predisposition for Deficiency in Health Functions Using SNP Analysis”, which is herein incorporated by reference in its entirety. More specifically, upon performing a SNP genotyping assay of a biological sample collected from an individual, the individual's genetic predisposition for DNA repair can be determined by using a specific DNA repair SNP panel which comprises predetermined DNA repair identifier SNPs. Such a SNP analysis determines and identifies an individual's genetic predisposition for DNA repair as normal, sub-normal, and deficient. The individual whose genetic predisposition for DNA repair is considered deficient is more likely to develop clinical conditions directly or indirectly related to deficient DNA repair process. By providing a suitable supplement composition based on an individual's genetic predisposition, the method of the present invention can more effectively assist in enhancing DNA repair and reducing the likelihood of the individual in developing clinical conditions caused by deficient DNA repair process.

In a further aspect, the method of the present invention provides a suitable supplement composition based on an individual's genetic predisposition for DNA repair. In one embodiment, an individual whose genetic predisposition for DNA repair is sub-normal is recommended to take one tablet of Supplement Composition A of Example 1 daily. In another embodiment of the present invention, an individual whose genetic predisposition for DNA repair is deficient is recommended to take two tablets of Supplement Composition B of Example 2 daily, or two tablets of Supplement Composition C of Example 3 daily. By providing a suitable supplement composition based on an individual's genetic predisposition, the method of the present invention can more effectively assist in enhancing the individual's DNA repair process and reducing the likelihood of the individual in developing clinical conditions related to DNA damages.

The following examples are illustrative of the invention and are in no way to be interpreted as limiting the scope of the invention, as defined in the claims.

### EXAMPLE 1

A composition of the following formulation was prepared in tablet form, including pharmaceutically acceptable excipients, by standard methods known to those of ordinary skill in the art:

TABLE 1
Supplement Composition A

| | Amount Per Serving | |
|---|---|---|
| Contents | (in one tablet) | |
| Beta-carotene | 5,000 | IU |
| Niacinamide | 150 | mg |
| Zinc citrate | 10 | mg |
| D-ribose | 150 | mg |
| Activar AC-11 ® | 175 | mg |

Other ingredients include: Dicalcium phosphate, microcrystalline cellulose, croscarmellose sodium, stearic acid, magnesium stearate, silica, and pharmaceutical glaze. In the example, the tablet has a weight from about 700 mg to about 900 mg.

### EXAMPLE 2

A composition of the following formulation was prepared in tablet form, including pharmaceutically acceptable excipients, by standard methods known to those of ordinary skill in the art:

TABLE 2
Supplement Composition B

| | Amount Per Serving | |
|---|---|---|
| Contents | (in two tablets) | |
| Beta-carotene | 5,000 | IU |
| Niacinamide | 250 | mg |
| Zinc citrate | 10 | mg |
| Indole-3-carbinol | 200 | mg |
| Resveratrol | 20 | mg |
| Activar AC-11 ® | 175 | mg |

Other ingredients include: Dicalcium phosphate, microcrystalline cellulose, croscarmellose sodium, stearic acid, magnesium stearate, silica, and pharmaceutical glaze. In the example, the tablet has a weight from about 700 mg to about 900 mg.

### EXAMPLE 3

A composition of the following formulation was prepared in tablet form, including pharmaceutically acceptable excipients, by standard methods known to those of ordinary skill in the art:

TABLE 3
Supplement Composition C

| | Amount Per Serving | |
|---|---|---|
| Contents | (in two tablets) | |
| Beta-carotene | 5,000 | IU |
| Niacinamide | 250 | mg |
| Zinc citrate | 10 | mg |
| D-ribose | 250 | mg |
| Indole-3-carbinol | 200 | mg |
| Resveratrol | 20 | mg |
| Activar AC-11 ® | 175 | mg |

Other ingredients include: Dicalcium phosphate, microcrystalline cellulose, croscarmellose sodium, stearic acid, magnesium stearate, silica, and pharmaceutical glaze. In the example, the tablet has a weight from about 700 mg to about 900 mg.

While the present invention has been described in detail and pictorially shown in the accompanying drawings, these should not be construed as limitations on the scope of the present invention, but rather as an exemplification of preferred embodiments thereof. It will be apparent, however, that various modifications and changes can be made within the spirit and the scope of this invention as described in the above specification and defined in the appended claims and their legal equivalents.

What is claimed is:

1. A supplement composition for enhancing DNA repair process, comprising effective amounts of:
(a) a carotenoid compound;
(b) a nicotinamide compound;
(c) a zinc compound;
(d) D-ribose; and
(e) a water soluble extract of an Uncaria species.

2. The supplement composition of claim 1, wherein said carotenoid compound is at least one selected from the group consisting of alpha carotene, beta carotene, gamma carotene, lycopene and combination thereof.

3. The supplement composition of claim 1, wherein said nicotinamide compound is at least one selected from the group consisting of nicotinamide, niacin, tryptophane, nicotinamide-adenine dinucleotide (NAD), reduced form of NAD (NADH), NAD phosphate (NADP), reduced form of NADP (NADPH) and combination thereof.

4. The supplement composition of claim 1, wherein said zinc compound is one or more zinc salts.

5. The supplement composition of claim 1 further comprising effective amounts of a glucosinolate compound and resveratrol.

6. The supplement composition of claim 5, wherein said glucosinolate compound is at least one selected from the group consisting of indole-3-carbinol, and isothiocyanates.

7. The supplement composition of claim 1 further comprising pharmaceutically acceptable excipients.

8. The supplement composition of claim 1, wherein said supplement composition is in a form for oral administration.

9. A supplement composition for enhancing DNA repair process, comprising in one dosage:
(a) from about 2,500 IU to about 10,000 IU of beta carotene;
(b) from about 75 mg to about 500 mg of nicotinamide;
(c) from about 5 mg to about 20 mg of zinc citrate;
(d) from about 75 mg to about 500 mg of D-ribose; and
(e) from about 85 mg to about 350 mg of a water soluble extract of an Uncaria species.

10. The supplement composition of claim 9 further comprising:
(f) from about 100 mg to about 400 mg of Indole-3-carbinol; and
(g) from about 10 mg to about 40 mg of resveratrol.

11. The supplement composition of claim 9 further comprising pharmaceutically acceptable excipients.

12. The supplement composition of claim 9, wherein said supplement composition is in a form for oral administration.

13. A supplement composition for enhancing DNA repair process, comprising in one dosage:
(a) from about 2,500 IU to about 10,000 IU of beta carotene;
(b) from about 75 mg to about 500 mg of nicotinamide;
(c) from about 5 mg to about 20 mg of zinc citrate;
(d) from about 100 mg to about 400 mg of Indole-3-carbinol;
(e) from about 10 mg to about 40 mg of resveratrol; and
(f) from about 85 mg to about 350 mg of a water soluble extract of an Uncaria species.

14. The supplement composition of claim 13 further comprising from about 75 mg to about 500 mg of D-ribose.

15. The supplement composition of claim 13 further comprising pharmaceutically acceptable excipients.

16. The supplement composition of claim 13, wherein said supplement composition is in a form for oral administration.

17. A method for enhancing DNA repair process comprising orally administering a supplement composition to a person daily in a dosage comprising from about 2,500 IU to about 10,000 IU of beta-carotene, from about 75 mg to about 500 mg of niacinamide, from about 5 mg to about 20 mg of zinc citrate, from about 75 mg to about 500 mg of D-ribose, and from about 85 mg to about 350 mg of water soluble extract of an Uncaria species.

18. The method of claim 17, wherein said supplement composition further comprises in said dosage from about 100 mg to about 400 mg of indole-3-carbinol and from about 10 mg to about 40 mg of resveratrol.
